(19) United States
(12) Patent Application Publication
(10) Pub. No.: US 2018/0185276 A1
(43) Pub. Date: Jul. 5, 2018

(54) CONTACT LENSES CARRYING MITOCHONRIALLY-TARGETED ANTIOXIDANTS

(71) Applicant: Anton Dmitrievich PETROV, Woodland Hills, CA (US)

(72) Inventors: Anton Dmitrievich Petrov, Woodland Hills, CA, CA (US); Maxim Vladimirovich Skulachev, Moscow (RU); Vadim Neronovich Tashlitsky, Moscow (RU); Elena Mirovna Karger, Moscow (RU); Lawrence T Friedhoff, River Vale, NJ (US)

(73) Assignee: Mitotech S.A., Luxembourg (LU)

(21) Appl. No.: 15/561,784

(22) PCT Filed: Mar. 25, 2016
(86) PCT No.: PCT/US16/24234

§ 371 Date: Sep. 26, 2017

Related U.S. Application Data
(60) Provisional application No. 62/138,721, filed on Mar. 26, 2015.

(51) Int. Cl. A61K 9/00; A61K 31/66; A61K 31/36; A61K 31/352; A61K 31/473; A61K 31/4741; A61P 27/02

(52) U.S. Cl. CPC A61K 9/0051; A61K 31/66; A61K 31/36; C11D 3/0078; A61K 31/473; A61K 31/4741; A61P 27/02; A61K 31/352

(57) ABSTRACT

The present inventor has discovered how to adsorb a mitochondrially targeted compound (MTC) of general formula I on contact lenses and other matrices prior the application of lenses or other matrix on the eye surface. After such matrix is positioned on eye, MTC is translocated from lens into cornea cells of the eye providing cornea protective effect and thus reducing side effects of contact lens application.

### BACKGROUND OF THE INVENTION

### Field of the Invention

The invention relates to the field of biomedicine, more particularly to the field of ophthalmology.

### SUMMARY OF THE RELATED ART

Oxidative stress is believed to be one of the key reasons for the development of different eye pathologies. Mitochondria-addressed antioxidants that can be reduced and oxidized by the enzymes of the electron transport chain of mitochondria are one of the most effective antioxidants (see Skualchev V. P. (2005), IUBMB Life. 57:305-10; Skulachev V. P. (2007) Biochemistry (Mosc.) 72:1385-96; Antonenko Yu. N. et al. (2008) Biochemistry (Mosc.)73:1273-87).

A principle possibility of treating eye diseases with the mitochondria-addressed antioxidant SkQ1 was shown in application WO2008048134, and in more detail in a paper by Neroev et al., (2008) Biochemistry (Mosc.)73:1317-28. These sources report data on the treatment of some eye diseases with the aid of aqueous solutions of SkQ1. However, said mitochondrial antioxidants have some peculiarities that complicate their use in practice. One of the main peculiarities is the observation that the antioxidant efficiency of a compound depends on its dose and final concentration in mitochondria. At certain concentrations these substances can act as one of the most powerful prooxidants that can cause mitochondria to produce a significant amount of reactive oxygen species (Antonenko Yu N. et al. (2008) Biochemistry (Mosc.)73:1273-87; Doughan A. K. and Dikalov S. I. (2007) Antioxid Redox Signal. 9:1825-36). Some substances routinely used as part of numerous preparations, e.g., eye drops, accelerate degradation of SkQ1.

Application of contact lenses frequently leads to damage of cornea of the patient. This problem is especially important for people with dry eye syndrome (DES) who use contact lenses.

Thus, there is a need for delivery of effective concentrations of mitochondrially-targeted antioxidants to the eyes, especially in cases of people who wear contact lenses.

### BRIEF SUMMARY OF THE INVENTION

The present inventor has discovered how to adsorb a mitochondrially-targeted compound (MTC) of general formula I on contact lenses and other eye-contacting matrices prior the application of lenses or other matrices on the eye surface. After such a contact lens is positioned on the eye, MTC is translocated from the contact lens into cornea cells of the eye providing a cornea protective effect and thus reducing side effects of contact lens application. Adsorption of mitochondrially-targeted compound on a contact lens can be performed by several methods, as further described herein. One method is to soak lenses in a water-based solution of MTC during the process of lens manufacturing. Another method is addition of MTC to lens storage solution. Another aspect of this invention is a lens storage (or preserving) solution containing MTC of formula I.

### BRIEF DESCRIPTION OF THE DRAWINGS

FIG. 1 shows dynamics of SkQ1 concentration in intact lenses (diamonds) and lenses applied on rabbit eyes (squares).

FIG. 2 shows dynamics of SkQ1 concentration in rabbit cornea. FIG. 2A: SkQ1 applied via SkQ supplemented contact lens; FIG. 2B: SkQ1 applied in eye drops.

### DETAILED DESCRIPTION OF THE PREFERRED EMBODIMENTS

The present inventor has discovered how to adsorb a mitochondrially-targeted compound (MTC) of general formula I on contact lenses and other eye-contacting matrices prior the application of lenses or other matrices on the eye surface. After such a contact lens is positioned on the eye, MTC is translocated from lens into cornea cells of the eye providing a cornea protective effect and thus reducing side effects of contact lens application. Adsorption of a mitochondrially-targeted compound on a contact lens can be performed by several methods, as further described herein. One method is to soak lenses in a water-based solution of MTC during the process of lens manufacturing. Another method is addition of MTC to lens storage solution. Another aspect of this invention is a lens storage (or preserving) solution containing MTC of formula I.

MTC useful in the invention can be described by formula I:

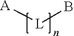

wherein A is an effector moiety, which is an antioxidant optionally having a following structure:

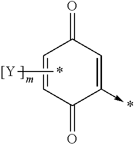

and/or reduced forms thereof,
wherein m is an integer from 1 to 3; each Y is independently selected from the group consisting of: lower alkyl, lower alkoxy; or two adjacent Y groups, together with carbon atoms to which they are attached, form a following structure:

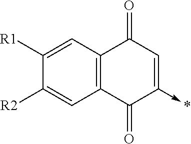

and/or reduced form thereof,
wherein R1 and R2 may be the same or different and are each independently lower alkyl or lower alkoxy;
L is a linker group, comprising:
a) straight or branched hydrocarbon chain which can be optionally substituted by one or more substituents and optionally contains one or more double or triple bonds;
b) a natural isoprene chain;
n is the number of carbon atoms in the linker, which is an integer from 1 to 40; and
B is a mitochondria targeting group comprising a lipophilic cation and a pharmacologically acceptable anion;
and/or solvates, salts, isomers or prodrugs thereof.

“Lower alkyl” and “lower alkoxy” mean, respectively, an alkyl or alkoxy group having 1 to 6 carbon atoms.

Specific non-limiting examples of MTC of formula I include:

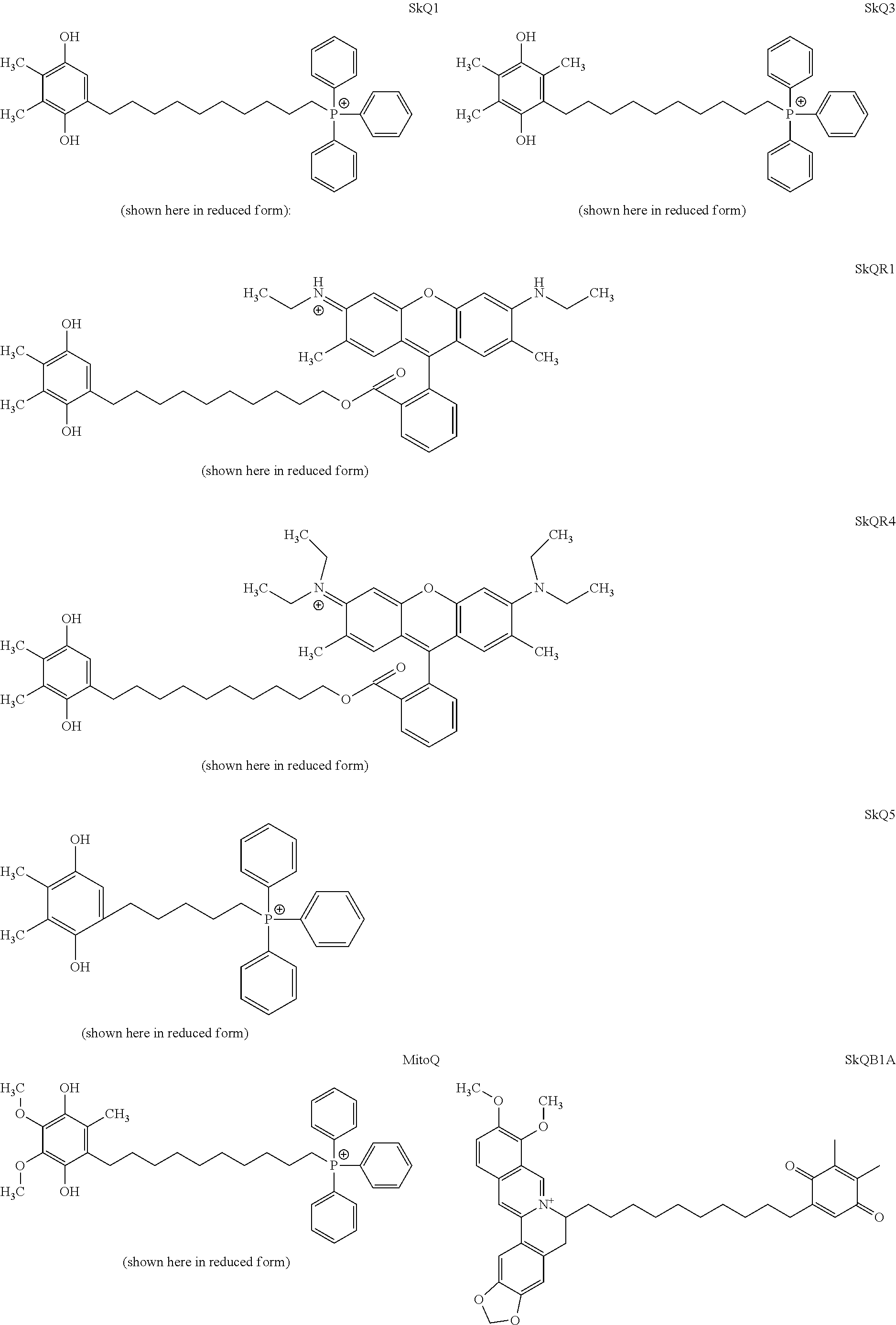

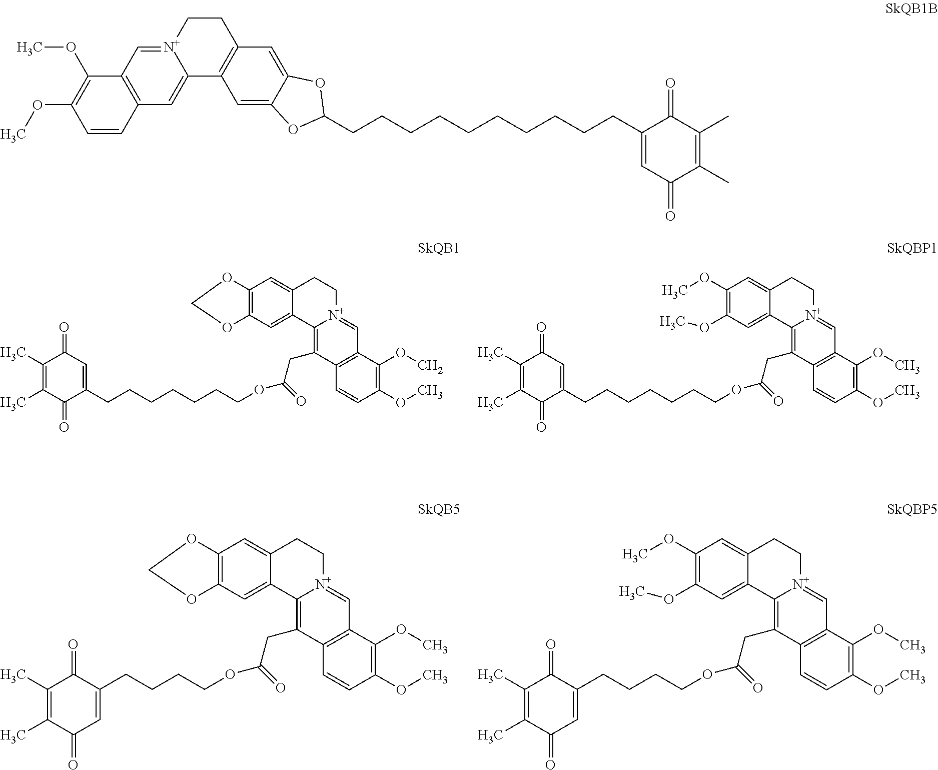

Most of the MTC can be present in reduced (quinole) form and oxidized (quinone) form. See for example the formula of oxidized form of SkQ1:

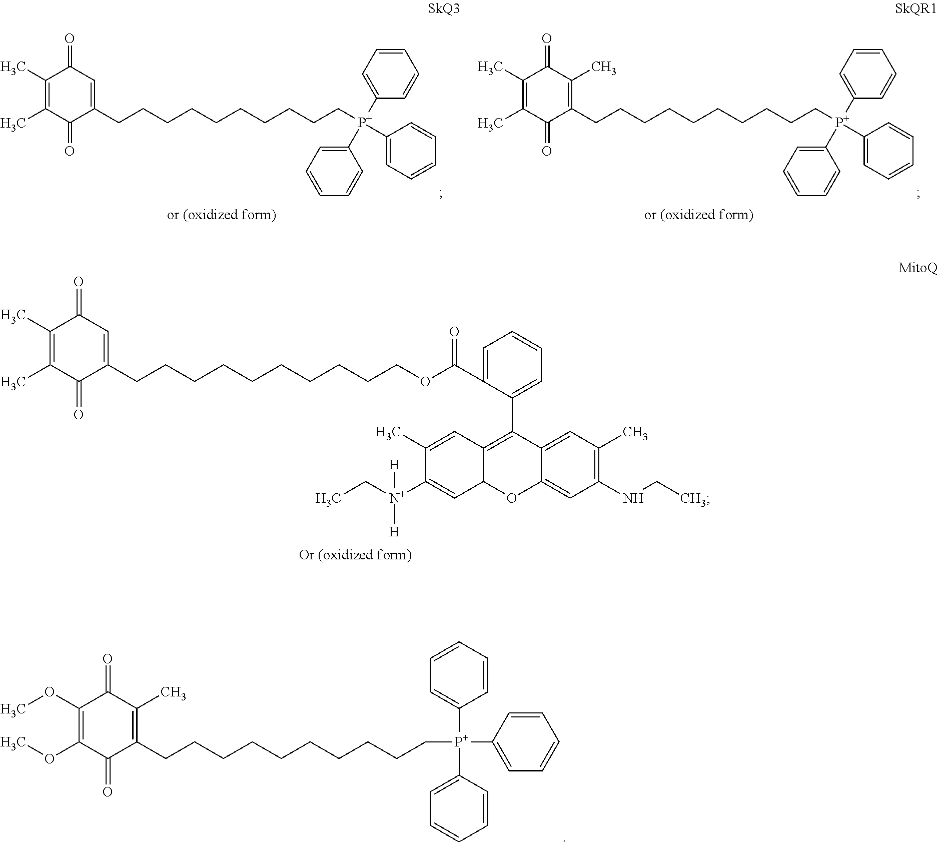 [SMILES: CC1=C(C)C(=O)C(CCCCCCCCCC[P+](c2ccccc2)(c2ccccc2)c2ccccc2)=CC1=O ; CC1=C(C)C(=O)C(CCCCCCCCCC[P+](c2ccccc2)(c2ccccc2)c2ccccc2)=C(C)C1=O ; CCNc1cc2c(cc1C)C(c1ccccc1C(=O)OCCCCCCCCCCC1=CC(=O)C(C)=C(C)C1=O)=C1C=C(C)C([NH2+]CC)=CC1O2 ; COC1=C(OC)C(=O)C(CCCCCCCCCC[P+](c2ccccc2)(c2ccccc2)c2ccccc2)=C(C)C1=O]

Oxidized forms of all other quinole-containing compounds are analogues, i.e. they differ from the reduced form by substitution of quinole with the corresponding quinone.

All of above compounds can be used in a form of salt with a pharmaceutically acceptable anions such as, without limitation: chloride, bromide, sulfate, phosphate, mesylate, citrate, acetate etc.

The main aspect of this invention is the discovery of the ability of MTC to reversibly bind to a contact lens. As shown in experimental example 1: (i) MTC can be adsorbed on the contact lens, (ii) MTC remains on the contact lens when the lens is rinsed with water, and (iii) MTC can be extracted back from the lens by strong solvent such as acetonitrile.

In the next experiment we have shown that adsorbed MTC stays in the lens in saline solution and it can be extracted from the lens by relatively hydrophobic solvent (see description in the experimental example 2).

In the next experiment we have shown that a MTC adsorbed on lens can be efficiently transferred from the lens into eye (see experimental example 3).

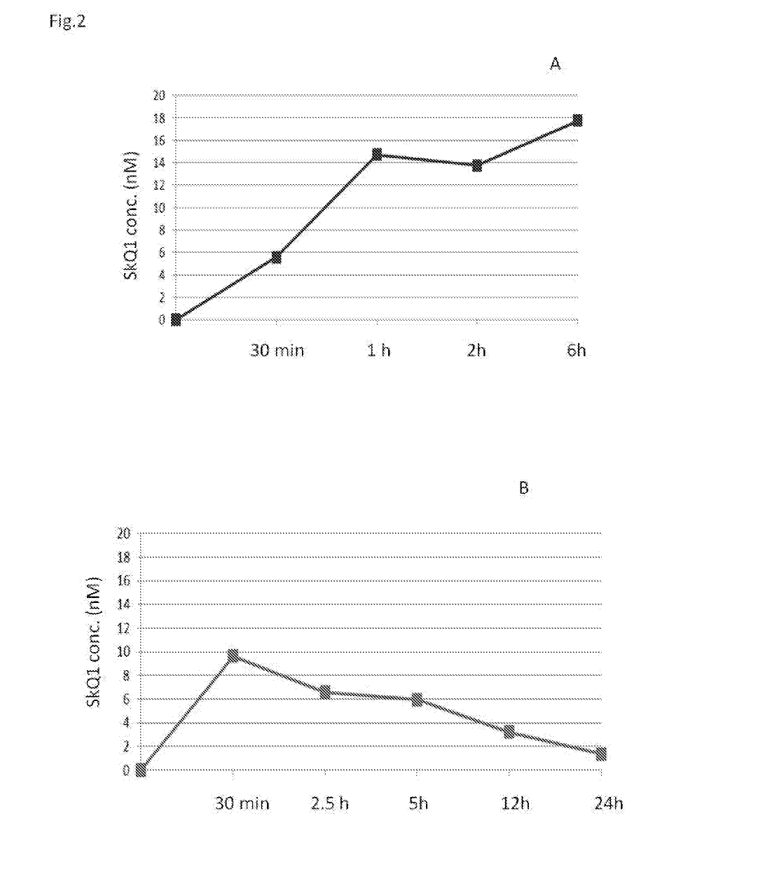

It can be concluded from comparison of FIG. 2A and FIG. 2B, that administration of MTC via a contact lens provides gradual and prolonged eye treatment compared to the usual way of administration (eye drops). It is also beneficial that the method of treatment (using the contact lens) does not require usage of lipophilic cation preservatives such as BAK, which is routinely used in eye drops with MTC (see WO2010143990). Thus one of the aspects of this invention is a new method of administration of MTCs into an eye comprising applying to the eye a polymer matrix, such as a contact lens, that has MTC reversibly adsorbed thereon.

It is also known that contact lenses may damage the eye, for example they may cause small injuries on cornea or conjunctiva. MTC, such as mitochondrially-targeted antioxidants can protect eye tissues from such damage, as well as stimulate regeneration and reduction of inflammation usually associated with such small injuries. Therefore, another aspect of this invention is a contact lens supplemented with MTC prior the use of such lenses. There are different ways of manufacturing such MTC-supplemented lenses. As shown in the experimental examples, MTC stays adsorbed on the lens in a lens storage solution. Thus, a lens can be supplied with MTC during a lens manufacturing process prior the final packaging of the lens. In this case it is important to adapt the manufacturing process to select a composition of compatible lens storage solution. For example lipophilic cation preservatives such as BAK should not be used in the storage solution because they can prevent adsorption of MTC on the contact lens or displace MTC from the contact lens. In the case of a disposable (single use) contact lens, the compatible solution is inside the lens package (for example, inside a blister). In the case of multiple use contact lenses, the compatible solution is used to clean the contact lens or store the contact lens when not in use. Thus, another aspect of this invention is a solution containing MTC which can be reversibly adsorbed on a contact lens when the contact lens is placed the solution.

One skilled in the art will recognize that SkQ1 was used in the experimental examples 1-3 as a typical representative of MTCs to illustrate the ability of MTCs generally to reversibly bind to a contact lens. Judging from the chemical structures of MTCs as described in Formula 1, it can be concluded that such other MTC compounds can be bound to lenses in a similar way. Additional, non-limiting, support for this conclusion is shown in experimental example 4.

One skilled in the art will also recognize that contact lenses were used as a non-limiting example of an MTC supplemented matrix in the above described experiments. In some other applications of the invention, other devices can used, as such matrices to deliver MTC into eye cells. A matrix can be used this way if (i) MTC can be reversibly adsorbed in the matrix and (ii) MTC can be transferred from the matrix into eye tissue when the matrix is contacted with the eye tissue. The experimental examples can be repeated using a test matrix to find out whether it is a matrix suitable for the delivery of MTC into eye.

Examples of such matrices carrying MTC are contact lenses, gels, dissolvable films, non-dissolvable films, lenses, polymer covers, implants, bandages, transplant materials, implantable devices including plumps, drainages, stents, sponges and the like.

Another aspect of the invention is a method of delivery of MTC into an eye tissue comprising reversible absorption of MTC on the matrix followed by transfer of MTC from the matrix into the eye tissue.

In another aspect of the invention, the MTC containing matrix is used to treat an existing eye disease. In another aspect of this invention the MTC containing matrix is used for prophylaxis of eye diseases. In some cases of treatment or prophylaxis, the MTC containing matrix is used daily. Such diseases include, without limitation, diseases of the cornea (such as dry eye syndrome), cataract, glaucoma, and macular degeneration.

The following examples are intended to further illustrate certain non-limiting aspects of the invention, and are not to be interpreted as limiting the scope of the invention.

### Example 1

100 ml of 2.5 μM or 25 μM SkQ1 initial solutions were prepared in dark glass vials. A polymer contact lens was placed in each solution and then incubated for 146 hours at +4° C. The lenses were then rinsed twice with distilled water and each lens was transferred into a dark glass test tube with 1 ml of 60% acetonitrile/water solution. The test tubes were shaken for 1 hour on +25° C. and the resulting content of SkQ1 in acetonitrile solution was analyzed by LCMS/MS. The results of this analysis are shown in Table 1.

TABLE 1

| | SkQ1 | Amount of | Amount |
|---|---|---|---|
| | concentration | SkQ1 found in | of SkQ1 found in |
| | in initial | contact lens at the end | contact lens at the end of |
| No | solution, μM | of experiment, μg | experiment, nmoles |
| 1 | 2.5 | 27.2 | 44.1 |
| 2 | 25 | 201.9 | 327.2 |

This experiment demonstrates that MTC can be reversibly adsorbed on a contact lens and then transferred in acetonitrile. In this experiment acetonitrile served as a model of cornea surface.

### Example 2

Lens pre-wash: soft disposable contact lenses were thoroughly rinsed with saline solution (0.9% NaCl) and incubated in this solution overnight.
Preparation of 2.5 μM SkQ1 solution: 4 mL of saline solution was placed in dark glass vials. 40 of 500 μM SkQ1 solution in 50% ethanol was added into each vial yielding 2.5 μM SkQ1 solution. Solution in the vial was thoroughly mixed and then incubated overnight to yield A1 series solution. Then aliquots of A1 series were collected from these vials.

During the experiment a pre-washed contact lens was placed into a glass vial with 2.5 uM SkQ1 solution and incubated at +4° C.-+8° C. for 4 days. Then the contact lenses were removed from the 2.5 μM SkQ1 solution, rinsed with saline solution and placed into fresh saline solution and incubated overnight at +4° C.-+8° C. to yield A2 series solution. Aliquots of the A2 series solution were collected from the vials where lenses were incubated in 2.5 μM SkQ1 solution.

After the above described overnight lens incubation in saline solution, lenses were placed in vials containing 500 μL of 50% acetonitrile and mixed during 1 hour at +25° C. Then aliquots of this acetonitrile solution was collected (A3 series).

The content of SkQ1 in the collected aliquots was analyzed using LCMS/MS. The results of this analysis are presented in Table 2.

TABLE 2

| | SkQ1 concentration found, | |
|---|---|---|
| | μg/ml | Calculated total SkQ1 amount in |
| Samples | M ± SE (n = 9) | the vial, μg, M ± SE (n = 9) |
| A1 series | 0.97 ± 0.03 | 3.69 ± 0.11 |
| A2 series | 0.1 ± 0.04 | 0.19 ± 0.07 |
| A3 series | 9.9 ± 0.33 | 2.5 ± 0.08 (=total SkQ1amount |
| | | extracted from the lens) |

These results show that the SkQ1 amount dissolved in the saline was significantly reduced after the placement of the contact lens into this solution (reduction from 3.69 μg to 0.19 μg). Then some significant amount (2.5 μg) of SkQ1 was extracted from the lens by 50% acetonitrile, but not by saline. Thus SkQ1 was adsorbed from saline solution in the lens and then extracted back by more hydrophobic solution.

### Example 3

This experiment demonstrates that an MTC (for example, SkQ1) can be adsorbed on a contact lens and then efficiently transferred from this lens into eye of the rabbit. The experiment was performed as follows: contact lenses were supplemented with SkQ1 as described in Example 2, then some lenses were placed on rabbit eyes and some lenses left aside in saline solution. At certain time points (see below in the table 3) contact lenses were removed from rabbits' eyes (experimental lenses) or saline solution (control lenses) and such rabbits were euthanized and their cornea were taken for SkQ1 content analysis. Then SkQ1 was extracted from the collected lenses and cornea samples using 50% acetonitrile solution and the SkQ1 amount in the extracts was determined using LCMS/MS. The concentrations are shown in Table 3.

TABLE 3
SkQ1 concentration in control lenses (intact lenses kept in
saline solution)

| | Sample | SkQ1 conc., | SkQ1 amount, |
|---|---|---|---|
| Time point | number | μg/mL | nmoles/sample |
| 30 min | 3 | 5.690 | 4.611 |
| 1 hour | 6 | 6.129 | 4.966 |
| 2 hours | 9 | 6.302 | 5.107 |
| 6 hours | 15 | 5.052 | 4.377 |

SkQ1 concentration in experimental lenses (lenses taken from rabbits eyes)

| | Sample | SkQ1 conc., | SkQ1 amount, | Average |
|---|---|---|---|---|
| Time point | number | μg/mL | nmoles/sample | nmoles/sample |
| 30 min | 1 | 2.868 | 2.324 | 2.38 |
| | 2 | 3.006 | 2.436 | |
| 1 hour | 4 | 3.339 | 2.706 | 2.65 |
| | 5 | 3.200 | 2.593 | |
| 2 hours | 7 | 2.77 | 2.244 | 2.279 |
| | 8 | 2.854 | 2.313 | |
| 6 hours | 13 | 1.448 | 1.174 | 1.114 |
| | 14 | 1.229 | 1.053 | |

SkQ1 concentration in the cornea

| | Sample | | ng per g of | | Aver. nM |
|---|---|---|---|---|---|
| Time point | number | ng/mL | cornea | nM in cornea | in cornea |
| 30 min | 17 | 0.64 | 3.84 | 6.22 | 5.59 |
| | 18 | 0.51 | 3.06 | 4.96 | |
| 1 hour | 19 | 1.56 | 9.36 | 15.17 | 14.73 |
| | 20 | 1.47 | 8.82 | 14.30 | |
| 2 hours | 21 | 1.46 | 8.76 | 14.20 | 13.76 |
| | 22 | 1.37 | 8.22 | 13.32 | |
| 6 hours | 25 | 2.85 | 17.1 | 27.72 | 17.75 |
| | 26 | 0.8 | 4.8 | 7.80 | |

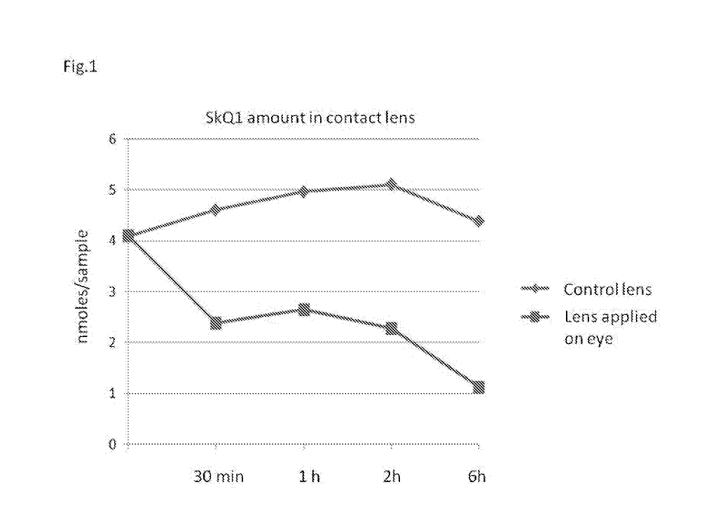

These results demonstrate that the amount of SkQ1 in the contact lens placed on the eye decreased with time (see lower line on FIG. 1), while the SkQ1 concentration stayed stable (with slight variation) on the intact control lenses left in saline. At the same time, the concentration of SkQ1 gradually increased in the rabbits' eye cornea (see FIG. 2A). Thus SkQ1 was efficiently transferred from the experimental lens into the eye. In the control experiment, SkQ1 was administered into the rabbit eye by instillation of eye drops containing 1.55 μg/mL of SkQ1. At certain time points, samples of rabbit cornea were taken and analyzed for SkQ1 content using LCMS/MS. The resulting graph is shown in FIG. 2B. It can be seen that, in this way of administration, SkQ1 concentration is maximum at 30 min and then decreased with time. Thus, the mode of accumulation of SkQ1 in the cornea is different when SkQ1 is administered via contact lenses (compare graphs on FIG. 2A and FIG. 2B).

### Example 4

This experiment demonstrates that different MTCs (i.e. having different molecular structures) can be used for supplementation of contact lenses. To demonstrate this, it is shown that SkQ1, SkQ3 and SkQB1 can be adsorbed on a matrix from saline solution. It must be noted that SkQ3 differs from SkQ1 by the antioxidant moiety (A moiety in the formula I—see above), while SkB1 differs from SkQ1 by both the linker (L moiety) and targeting moiety (B moiety). So in this experiment, variations in all 3 parts of MTC molecule were successfully used.

The experiment was conducted as follows: 990 μl of phosphate buffered saline (PBS) or 50% water-ethanol solution (SkQ adsorption in 50% ethanol is negligible) was placed in dark glass chromatography vials (Waters, p/n 186000847c). 10 μl of 25 μM SkQ solution in ethanol was then added to each vial, yielding a 250 nM solution. The vials were vigorously mixed and the SkQ concentration in each vial (100 μl samples) was analyzed by HPLC using a Luna C 18 (2), Phenomenex, 250 MM ′4.6 mm column with a sorbent particle size of 5 μm. The liquid phase used was acetonitrile:water (56:44), and the flow speed was 1 ml/min. at 25° C. UV detection was performed at A260.

The concentration of SkQ1 in PBS was 51.7% of the concentration of SkQ1 in 50% ethanol. In the case of SkQ3 the concentration of SkQ3 in PBS was 29.6%, and in the case of SkQB1 the concentration of SkQB1 in PBS was 63.8%. These results indicate that all MTCs used in this experiment bind to the matrix in a way similar to SkQ1.

What is claimed is:

1. A matrix for contacting the eye, the matrix containing one or more mitochondrially-targeted compound of general formula I: 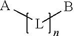
wherein A is an effector moiety, which is antioxidant having a following structure: 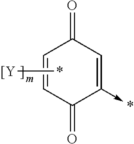
and/or reduced forms thereof,
wherein m is an integer from 1 to 3; each Y is independently selected from the group consisting of: lower alkyl, lower alkoxy; or two adjacent Y groups, together with carbon atoms to which they are attached, form a following structure: 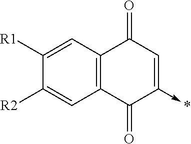
and/or reduced forms thereof,
wherein R1 and R2 may be the same or different and are each independently lower alkyl or lower alkoxy;
L is a linker group, comprising:
a) straight or branched hydrocarbon chain which can be optionally substituted by one or more substituents and optionally contains one or more double or triple bonds;
b) a natural isoprene chain;
n is the number of carbon atoms in the linker, which is an integer from 1 to 40; and
B is a mitochondria targeting group comprising a lipophilic cation a pharmacologically acceptable anion;
and/or solvates, salts, isomers or prodrugs thereof.

2. The matrix according to claim 1 which is a contact lens.

3. The matrix according to claim 1 which is an eye gel.

4. The matrix according to claim 1 which is a dissolvable or non-dissolvable film

5. The matrix according to claim 1 which is an implant or a part of implantable device.

6. An MTC-containing solution for storage of a matrix that adsorbs the MTC.

7. A lens storage solution according to claim 6 containing one or more mitochondrially-targeted compound of formula I.

8. A method of manufacturing of a matrix comprising a step of reversible absorption of a mitochondrially targeted compound of formula I on the matrix.

9. A method of manufacturing of a lens comprising a step of reversible absorption of a mitochondrially targeted compound of formula I on a lens.

10. A method of treatment or prophylaxis of an eye disease comprising applying to an eye one or more matrix or solution according to claim 1, 2, 3, 4, 5, or 6.
